(19) United States
(12) Patent Application Publication
(10) Pub. No.: US 2025/0164112 A1
(43) Pub. Date: May 22, 2025

(54) wARRANGEMENT WITH SUPPLY LINES

(71) Applicant: Siemens Aktiengesellschaft, München (DE)

(72) Inventors: Frank Sperrfechter, Ötigheim (DE); Simon Weida, Göcklingen (DE); Mathias Baader, Landau (DE); Heike Bindig, Ettlingen (DE)

(73) Assignee: Siemens Aktiengesellschaft, München (DE)

(21) Appl. No.: 18/946,018

(22) Filed: Nov. 13, 2024

(30) Foreign Application Priority Data
Nov. 22, 2023 (EP) 23211581.6

(51) Int. Cl. F23J 13/02; H02G 3/04

(52) U.S. Cl. CPC F23J 13/02; H02G 3/0412

(57) ABSTRACT

An arrangement comprises at least one first electrically conductive supply line, a sheathing, and a rod-shaped insulating body. The sheathing has a first end and a second end and the first end is different from the second end and the first end is opposite the second end and the sheathing has a first opening at the first end and a second opening at the second end. The first supply line runs from the first end to the second end through the arrangement and defines an axis. The rod-shaped insulating body comprises a passage through the rod-shaped insulating body and a first section of the supply line runs in the passage through the rod-shaped insulating body.

### CROSS-REFERENCE TO RELATED APPLICATIONS

This application claims priority to EP Application Serial No. 23211581.6 filed Nov. 22, 2023, the contents of which are hereby incorporated by reference in their entirety.

### TECHNICAL FIELD

The present disclosure relates to combustion apparatus. Various embodiments include methods and/or systems with supply lines for use in a combustion apparatus.

### BACKGROUND

Industrial processes use energy conversion through combustion to generate steam and/or heat for an industrial process. For this purpose, a flame from a heat generator burns in the combustion chamber of a combustion apparatus during operation. The heat generator exchanges the thermal energy of the hot fuel gases in another fluid such as for example water. The warm water is used for example to operate a hot water heating system and/or to heat up drinking water. It is possible using the thermal energy of the hot fuels and/or fuel gases to heat up a product for example in an industrial process. The heat generator may be part of a system having a power-heat coupling, for example a motor of such a system. Moreover, the heat generator can serve to heat up water in a system for the extraction of lithium and/or lithium carbonate. The waste gases are discharged from the combustion chamber, for example via a waste gas stack and/or a flue gas stack and/or a chimney. A sensor element which can, for example, record an oxygen concentration is then arranged in the waste gas stack and/or flue gas stack and/or chimney.

Some such processes involve the operation of a furnace or boiler. While the combustion represents a cost-effective energy conversion, attempts are frequently made to maximize the combustion efficiency within a process. Maximizing combustion efficiency is, among other things, a consequence of the resulting waste gases and/or flue gases which leave the system. These exhaust gases and/or flue gases are sometimes subject to regulations regarding the emission of harmful gases. Consequently, one aim of optimization is to maximize the combustion efficiency of existing furnaces and/or boilers. This is accompanied by a reduction in the production of greenhouse gases and other harmful by-products.

Another goal is to optimize for different fuels and/or fuel gases. In particular, this involves fuels and/or fuel gases which comprise hydrogen gases. The fuels and/or fuel gases in question may comprise more than twenty percent hydrogen by volume at 293 Kelvin. In some cases, the proportion of hydrogen gas at 293 Kelvin is fifty percent by volume or even seventy percent by volume.

Combustion efficiency can be optimized by controlling the oxygen content in the waste gases and/or flue gases originating from a combustion process. This largely ensures the oxidation of the combustion by-products.

In-situ or in-process analysis devices can be used to monitor and/or optimize and/or control an ongoing combustion process. Common analysis devices include a sensor unit. The sensor unit is heated to high temperatures. It operates directly in the combustion zone or close to the combustion zone of the furnace or boiler.

Known analysis devices typically use a zirconium dioxide-based oxygen sensor. The oxygen sensor is located at one end of a probe that is inserted into a flue gas stream. As the waste gas and/or flue gas flows into the analysis device, it diffuses through a filter or diffuser into the vicinity of the zirconium dioxide-based oxygen sensor. There are no pumps and/or other flow-inducing apparatuses which are used to direct the sample flow into the analysis device. Instead, the gas passively passes through the diffuser. The sensor provides an electrical signal that indicates the amount of oxygen present in the waste gas and/or flue gas.

The zirconium dioxide-based oxygen sensor provides a potentiometric indication. The potentiometric indication is considered a reliable oxygen measurement in combustion environments. It enables efficient and/or safe process control. Typically, a single probe is inserted into the process, for example into the waste gas stack and/or flue gas stack and/or chimney. Percentage oxygen measurement is used in order to optimize combustion efficiency in small boilers and/or furnaces. In large systems, the operator often encounters waste gas stratification and/or flue gas stratification. The waste gas stratification and/or flue gas stratification comprises a multiplicity of layers, each with a different oxygen concentration.

To obtain stratification information, operators can install multiple probes in the waste gas stack and/or in the flue gas stack and/or in the chimney for efficient and safe operation. In individual cases, up to sixteen such probes can be installed.

High demands are placed not only on the sensor unit in terms of temperature resistance and resistance to chemical decomposition. The supply lines to the sensor unit must also fulfill the same or similar requirements. In particular, supply lines that make electrical contact with the sensor unit must be temperature-resistant and resistant to chemical decomposition.

A published utility model CN2685875Y from China deals with an integral smoke analysis device based on zirconium dioxide. The document CN2685875Y discloses an analysis device having a measuring tip. A zirconium dioxide-based sensor, a heater and a temperature sensor are attached to a first end of the measuring tip. Two gas connections and a housing are located at a second end of the measuring tip. An outlet protrudes from the housing. CN2685875Y discloses measures for protecting the supply lines to the zirconium dioxide-based sensor and for protecting the supply lines to the temperature sensor. The measures include a measuring tube, which is attached to a flange, and a rain shield.

An international patent application WO2022/064271A1 deals with an in-situ analysis device with averaging. WO2022/064271A1 discloses an analysis device having a measuring tip. The measuring tip has a first and a second end. A plurality of openings is located between the first and second end of the measuring tip. A sensor unit and a flange for mounting the analysis device are located near the second end of the measuring tip. According to WO2022/064271A1, each sensor element requires supply lines and/or signal lines.

A patent application DE102012211039A1 deals with a gas sensor for soot. Here a sensor unit is arranged in a protective tube. Furthermore, a mounting connecting piece with a reduced diameter compared to a diameter of a housing is provided.

The patent U.S. Pat. No. 6,015,533A discloses a sensor connected by a plurality of electrical cables. A cable harness supports the electrical cables within a tube. A wall delimits the waste gas chamber. The cables and the cable harness are arranged outside the wall in such a manner that the cables and the cable harness and its sheathing are not exposed to the waste gas flow.

A patent application US2010/050738A1 deals with a sensor arrangement with a thermally insulating housing. The arrangement is divided into a first and a second cylindrical section. A flange is located between the first and second cylindrical sections. The first cylindrical section comprises an inlet opening and an outlet opening. The second cylindrical section surrounds a plurality of cables. Similar to the cable harness of U.S. Pat. No. 6,015,533A, the cables of US2010/050738A1 are guided by means of a cable support sleeve. The cable support sleeve comprises passages for the individual cables.

A European patent application EP4236640A1 deals with a holder for a printed circuit board. The holder of EP4236640A1 comprises at least one tubular fastening element. At least one electrical conductor is arranged within the at least one tubular fastening element. The at least one tubular fastening element is guided through a first opening of the holder.

A patent application CN115791931A discloses a packing structure for an oxygen sensor with regard to the industrial automation of a combustion process. In particular, CN115791931A discloses a ceramic ring within a tube. According to CN115791931A, a ceramic insulating piece has a plurality of feedthroughs. Platinum wires run in these feedthroughs.

### SUMMARY

The teachings of the present disclosure include arrangements with one or multiple supply lines which enables gas analysis on a combustion apparatus. For this purpose, the arrangement is to be designed such that it can withstand the thermal as well as mechanical and chemical stresses during operation. For example, some embodiments of the teachings herein include an arrangement comprising at least one first electrically conductive supply line (1a, 1b, 1c), a sheathing (2) and a rod-shaped insulating body (3) which is made of an electrically insulating and temperature-resistant material; wherein the sheathing (2) has a first end (4a) and a second end (4b) and the first end (4a) is different from the second end (4b) and the first end (4a) is opposite the second end (4b) and the sheathing (2) has a first opening at the first end (4a) and a second opening at the second end (4b); wherein the at least one first supply line (1a, 1b, 1c) runs from the first end (4a) to the second end (4b) through the arrangement and defines an axis; wherein the rod-shaped insulating body (3) comprises at least one passage through the rod-shaped insulating body (3) and a first section of the at least one supply line (1a, 1b, 1c) runs in the at least one passage through the rod-shaped insulating body (3); wherein the at least one first supply line (1a, 1b, 1c) has a first length between the first opening and the second opening; wherein the first section of the at least one supply line (1, 1b, 1c) has a second length in the at least one passage through the rod-shaped insulating body (3); wherein the rod-shaped insulating body (3) comprises an outer surface radially outwards from the axis and the sheathing (2) comprises an inner surface outwards from the axis, wherein the outer surface of the rod-shaped insulating body (3) and the inner surface of the sheathing (2) each run parallel to the axis, so that the outer surface of the rod-shaped insulating body (3) and the inner surface of the sheathing (2) lie opposite one another; wherein the arrangement comprises a gap which has a size and is arranged between the outer surface of the rod-shaped insulating body (3) and the inner surface of the sheathing (2) and comprises spacers (5, 6a, 6b, 7a, 7b) so as to provide a spacing between the rod-shaped insulating body (3) and the sheathing (2), wherein the spacers (5, 6a, 6b, 7a, 7b) define the size of the gap; wherein the second length is smaller than the first length; and wherein the spacers (5, 6a, 6b, 7a, 7b) comprise a first ring (6a, 6b), which runs along a first closed curve around the rod-shaped insulating body (3) and adjoins the rod-shaped insulating body (3) and adjoins the sheathing (2).

In some embodiments, the spacers (5, 6a, 6b, 7a, 7b) are designed so as to provide a spacing between the outer surface of the rod-shaped insulating body (3) and the inner surface of the sheathing (2) and define the size of the gap.

In some embodiments, the at least one first supply line (1a, 1b, 1c) runs from the first end (4a) to the second end (4b) in a straight line through the arrangement and defines the axis.

In some embodiments, the spacers (5, 6a, 6b, 7a, 7b) comprise a surround body (5), and the surround body (5) comprises at least one first section, which runs perpendicular to the axis and adjoins the rod-shaped insulating body (3), and at least one second section, which runs parallel to the axis and adjoins the sheathing (2).

In some embodiments, the surround body (5) comprises at least one passage and a second section of the at least one supply line (1a, 1b, 1c) in the at least one passage runs through the surround body (5).

In some embodiments, the surround body (5) can be plugged onto the rod-shaped insulating body (3) in the axial direction outwards from the axis.

In some embodiments, the surround body (5) can be plugged into the sheathing (2) in the axial direction outwards from the axis.

In some embodiments, the surround body (5) has a first spacing to the first end (4a) and a second spacing to the second end (4b), wherein the first spacing of the surround body (5) from the first end (4a) is greater than the second spacing of the surround body (5) from the second end (4b).

In some embodiments, the rod-shaped insulating body (3) and the surround body (5) are each electrical insulators and at temperatures of 393 Kelvin each have a specific electrical resistance p of at least one megaohm·centimeter.

In some embodiments, the first ring (6a, 6b) has a first spacing to the first end (4a) and a second spacing to the second end (4b), wherein the first spacing of the first ring (6a, 6b) from the first end (4a) is greater than the second spacing of the first ring (6a, 6b) from the second end (4b).

In some embodiments, the surround body (5) comprises a recess and the first ring (6a, 6b) comprises a section which is arranged and/or runs in the recess of the surround body (5).

In some embodiments, the spacers (5, 6a, 6b, 7a, 7b) comprise a second ring (7a, 7b), which runs along a second closed curve around the rod-shaped insulating body (3) and adjoins the rod-shaped insulating body (3) and adjoins the sheathing (2) and is different from a or from the first ring (6a, 6b), and the second ring (7a, 7b) has a first spacing to the first end (4a) and a second spacing to the second end (4b), wherein the first spacing of the second ring (7a, 7b) from the first end (4a) is smaller than the second spacing of the second ring (7a, 7b) from the second end (4b).

As another example, embodiments include a combustion some apparatus comprising a combustion chamber and a structure selected from: an exhaust gas stack, a flue gas stack, a chimney; the structure is in fluid communication with the combustion chamber; the combustion apparatus comprises an arrangement as described herein; and at least one section (10b) of the arrangement is arranged within the structure.

In some embodiments, the structure comprises an outer wall (9) and the sheathing (2) of the arrangement is guided through the outer wall (9) in such a manner that the at least one section (10b) of the arrangement protrudes into the structure.

As another example, some embodiments include an arrangement comprising at least one first electrically conductive supply line (1a, 1b, 1c), a sheathing (2) and a rod-shaped insulating body (3) which is made of an electrically insulating and temperature-resistant material; wherein the sheathing (2) has a first end (4a) and a second end (4b) and the first end (4a) is different from the second end (4b) and the first end (4a) is opposite the second end (4b) and the sheathing (2) has a first opening at the first end (4a) and a second opening at the second end (4b); wherein the at least one first supply line (1a, 1b, 1c) runs from the first end (4a) to the second end (4b) through the arrangement and defines an axis; wherein the rod-shaped insulating body (3) comprises at least one passage through the rod-shaped insulating body (3) and a first section of the at least one supply line (1a, 1b, 1c) runs in the at least one passage through the rod-shaped insulating body (3); wherein the at least one first supply line (1a, 1b, 1c) has a first length between the first opening and the second opening; wherein the first section of the at least one supply line (1a, 1b, 1c) has a second length in the at least one passage through the rod-shaped insulating body (3) and the second length is smaller than the first length; wherein the rod-shaped insulating body (3) comprises an outer surface radially outwards from the axis and the sheathing (2) comprises an inner surface radially outwards from the axis, wherein the outer surface of the rod-shaped insulating body (3) and the inner surface of the sheathing (2) each run parallel to the axis, so that the outer surface of the rod-shaped insulating body (3) and the inner surface of the sheathing (2) lie opposite one another; wherein the arrangement comprises a gap which has a size and is arranged between the outer surface of the rod-shaped insulating body (3) and the inner surface of the sheathing (2), and comprises spacers (5, 6a, 6b, 7a, 7b) so as to provide a spacing between the rod-shaped insulating body (3) and the sheathing (2), wherein the spacers (5, 6a, 6b, 7a, 7b) define the size of the gap; wherein the spacers (5, 6a, 6b, 7a, 7b) comprise a surround body (5), and wherein the surround body (5) comprises at least one first section, which runs perpendicular to the axis and adjoins the rod-shaped insulating body (3), and at least one second section, which runs parallel to the axis and adjoins the sheathing (2).

### BRIEF DESCRIPTION OF THE DRAWINGS

Various features will become apparent to the person skilled in the art from the following detailed description of the disclosed non-limiting embodiments. The drawings which are attached to the detailed description can be described in short as follows.

FIG. 1 shows a schematic of an example a supply line arrangement incorporating teachings of the present disclosure; and

FIG. 2 shows a schematic of an example supply line arrangement as a feedthrough through a wall incorporating teachings of the present disclosure.

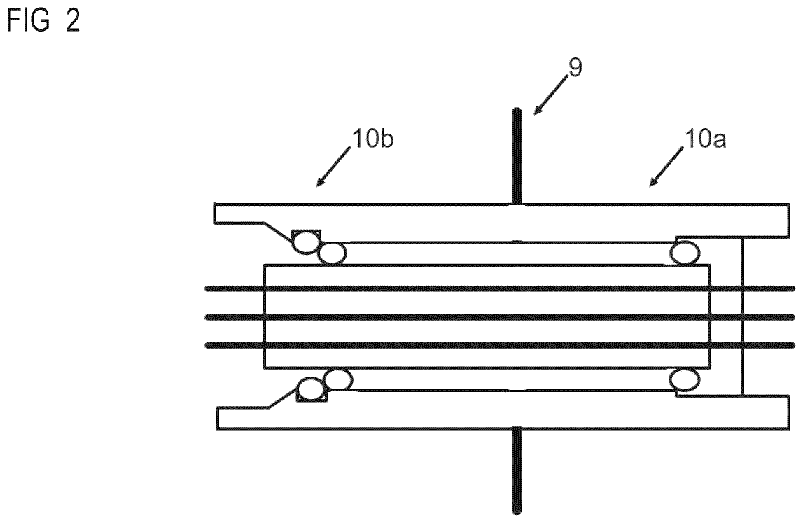

### DETAILED DESCRIPTION

Some examples of the teachings herein include arrangements comprising one or multiple supply lines. The one or multiple supply lines run through a rod-shaped insulating body. The one or multiple supply lines may run parallel to one another through a rod-shaped insulating body. The rod-shaped insulating body comprises for this purpose one or multiple passages for the one or multiple supply lines. The one or multiple supply lines are guided with the aid of one or multiple passages through the rod-shaped insulating body. In addition, the one or multiple supply lines are fixed with the aid of the one or multiple passages through the rod-shaped insulating body.

The one or multiple supply lines can be guided in particular through the rod-shaped insulating body in such a manner that the through-line is gas-tight. In some embodiments, the arrangement is gas-tight with respect to combustion gases from a combustion apparatus. This means that the one or multiple passages encompass the one or multiple supply lines in such a manner that gases, such as for example combustion gases, are prevented from passing through.

The one or multiple supply lines are electrically conductive. The one or multiple supply lines are made of a material with high temperature resistance. As a consequence, the arrangement with its supply line or its supply lines is suitable for use in a combustion apparatus.

The rod-shaped insulating body is made of an electrically insulating and temperature-resistant material. In some embodiments, the rod-shaped insulating body is highly resistant to chemical decomposition caused by combustion gases. For example, the rod-shaped insulating body can be made of a ceramic material.

A sheathing surrounds the rod-shaped insulating body and the one or multiple supply lines. The sheathing must also be temperature-resistant and resistant to chemical decomposition. For example, the sheathing can be made of corrosion-resistant steel. It is also important to ensure that the material of the sheathing is compatible with the material of an outer wall of a combustion apparatus. This is important if the sheathing is guided through the wall of a combustion apparatus.

There is a gap with a non-zero size between the sheathing and the rod-shaped insulating body. The gap can, for example, be filled with air and/or combustion gases, generally with a gas. The gap improves the thermal insulation of the rod-shaped insulating body and the supply lines from the sheathing.

Spacers provide a spacing between the rod-shaped insulating body and the sheathing. The spacers can comprise a surround body, in particular a cast body. The surround body, in particular the cast body, is arranged near one end of the sheathing and fixes the rod-shaped insulating body. The surround body, in particular the cast body, can also seal the arrangement gas-tight in the direction of that end.

One or multiple passages for the one or multiple supply lines are provided in the surround body, in particular in the cast body.

The spacers can also include one or multiple rings which provide a spacing between the rod-shaped insulating body and the sheathing.

The arrangement is suitable for installation in a combustion apparatus. The one supply line or the multiple supply lines are suitable for galvanically connecting to a sensor unit within the combustion apparatus. For this purpose, the one supply line or the multiple supply lines is or are galvanically connected at its end or at their ends to a sensor unit. A sensor unit inside the combustion apparatus can thus be electrically contacted.

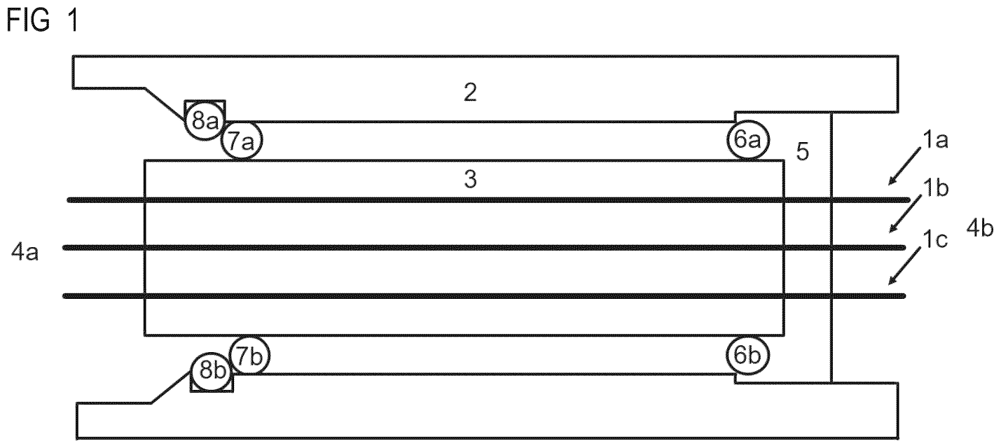

FIG. 1 illustrates an example arrangement of one or multiple supply lines 1a, 1b, 1c incorporating teachings of the present disclosure. The arrangement comprises a sheathing 2. The sheathing 2 can comprise for example a tubular sheathing. The sheathing 2 can be in particular a tubular sheathing. In a special embodiment, the sheathing 2 is cylindrical symmetrical around an axis which runs parallel to the supply lines 1a, 1b, 1c.

In some embodiments, the sheathing 2 is made of steel. In some embodiments, the sheathing 2 is made of corrosion-resistant steel. A sheathing 2 made of corrosion-resistant steel provides resistance to chemical degradation in an exhaust gas duct. In some embodiments, the sheathing 2 can be made of ferritic steel. In addition, the sheathing 2 can be made of austenitic steel.

In some embodiments, at least one of the supply lines 1a, 1b, 1c is resistant to high temperatures. It can therefore withstand the temperatures in an exhaust gas duct of a combustion apparatus such as a gas burner. In a high-temperature-resistant embodiment, at least one of the supply lines 1a, 1b, 1c comprises nickel wire. In some embodiments, at least one of the supply lines 1a, 1b, 1c comprises nickel wire. For example, at least one of the supply lines 1a, 1b, 1c may comprise an alloy which comprises more than seventy or more than seventy-five percent nickel by mass.

In some embodiments, all supply lines 1a, 1b, 1c are highly temperature resistant. Consequently, they withstand temperatures in an exhaust gas duct of a combustion apparatus such as for example a gas burner. In some high-temperature-resistant embodiments, all supply lines 1a, 1b, 1c comprise nickel wire. In some embodiments, all supply lines 1a, 1b, 1c comprise nickel wire. For example, all supply lines 1a, 1b, 1c can comprise an alloy which comprises more than seventy or more than seventy-five percent nickel by mass.

The supply lines 1a, 1b, 1c each have a diameter which is less than the smallest inside diameter of the sheathing 2. For example, the supply lines can have a diameter of 0.5 millimeters or one millimeter. Diameters which are greater than one millimeter are also possible. In some embodiments, all supply lines 1a, 1b, 1c have the same diameter. Identical diameters reduce the number of variants of the arrangement. With the number of variants, the risk of one of the variants failing during operation also decreases.

A rod-shaped insulating body 3 is arranged inside in the sheathing 2. The rod-shaped insulating body 3 is preferably a rod-shaped, electrical insulating body and/or a rod-shaped electrical insulation. In some embodiments, the rod-shaped insulating body 3 has a specific electrical resistance p of at least one megaohm·centimeter at temperatures of 873 Kelvin:

$$ρ > 1 MΩ \cdot cm at 873 Kelvin$$

The specific electrical resistance p at temperatures of 873 Kelvin may be greater than five megaohm·centimeters:

$$ρ > 5 MΩ \cdot cm at 873 Kelvin$$

Furthermore, the specific electrical resistance p at temperatures of 873 Kelvin may be greater than ten megaohm·centimeters:

$$ρ > 10 MΩ \cdot cm at 873 Kelvin$$

High specific resistances enable sufficient electrical insulation between the supply lines 1a, 1b, 1c. Any signal obtained from a sensor element that is connected to the supply lines 1a, 1b, 1c is therefore not distorted to any great extent.

In some embodiments, the rod-shaped insulating body 3 comprises ceramic, in particular aluminum oxide ceramic. In one particular embodiment, the rod-shaped insulating body 3 is made of ceramic, in particular aluminum oxide ceramic. In some embodiments, the aluminum oxide ceramic has a purity of more than 92 percent. In some embodiments, the aluminum oxide ceramic has a purity of more than 96 percent. In some embodiments, the aluminum oxide ceramic has a purity of more than 99 percent. Improved purity of the ceramic leads to more predictable behavior in terms of electrical insulation and mechanical strength.

In some embodiments, the rod-shaped insulating body 3 comprises a ceramic material based on magnesium silicate. In some embodiments, the rod-shaped insulating body 3 can comprise a ceramic material based on magnesium silicate. In some embodiments, the rod-shaped insulating body 3 comprises porcelain. In some embodiments, the rod-shaped insulating body 3 can comprise porcelain. In some embodiments, the rod-shaped insulating body 3 comprises porcelain. Moreover, the rod-shaped insulating body 3 can comprise porcelain.

The rod-shaped insulating body 3 has at least one passage for one of the supply lines 1a, 1b, 1c. In some embodiments, the rod-shaped insulating body 3 has at least as many passages as the arrangement has supply lines 1a, 1b, 1c. Moreover, the number of passages through the rod-shaped insulating body 3 can exceed the number of supply lines 1a, 1b, 1c. With a number of passages through the insulating body 3 that exceeds the number of supply lines 1a, 1b, 1c, further supply lines can be added at a later point in time. It is also possible for the same rod-shaped insulating body 3 to be used for embodiments with different numbers of supply lines 1a, 1b, 1c. The use of a rod-shaped insulating body 3 with a predetermined number of passages, which covers all embodiments, limits the number of variants. Consequently, the probability that one of the variants fails during the course of their operation reduces.

The passage or passages through the rod-shaped insulating body 3 each have a diameter. For example, the passage or passages through the rod-shaped insulating body 3 can have a diameter of at least 0.6 millimeters or of at least 1.1 millimeters. One or multiple passages through the rod-shaped insulating body 3 which have a diameter greater than 1.5 millimeters are also possible.

In the case of multiple passages through the rod-shaped insulating body 3, at least two passages through the rod-shaped insulating body 3 may have the same diameter. In some embodiments, all passages through the rod-shaped insulating body 3 have the same diameter. Identical diameters of the passages through the rod-shaped insulating body 3 lead to a smaller number of variants. Consequently, the probability that one of the variants fails during the course of their operation reduces. In addition, the outlay for the production of the rod-shaped insulating body 3 reduces.

At least one passage through the rod-shaped insulating body 3 can comprise a hole, for example a hole with a round cross-section. In particular, at least one passage through the rod-shaped insulating body 3 can be a hole, for example a hole with a round cross-section. Moreover, all passages through the rod-shaped insulating body 3 can each comprise a hole, for example a hole with a round cross-section. In addition, all passages through the rod-shaped insulating body 3 can each be a hole, for example a hole with a round cross-section. The design of the passages through the rod-shaped insulating body 3 as holes renders it possible to use standard tools for producing the insulating body 3.

The sheathing 2 has a first end 4a and a second end 4b. The first end 4a of the sheathing 2 is different from the second end 4b of the sheathing 2. The first end 4a of the sheathing 2 is opposite the second end 4b of the sheathing 2.

The sheathing 2 has a first opening on its first end 4a. In some embodiments, the first opening has a round cross-section. The sheathing 2 has a second opening on its second end 4b. In some embodiments, the second opening has a round cross-section. The first opening of the sheathing 2 is different from the second opening of the sheathing 2. In some embodiments, both the first opening of the sheathing 2 and also the second opening of the sheathing 2 have a round cross-section.

The area of the cross-section of the first opening of the sheathing 2 is usually different from the area of the cross-section of the second opening of the sheathing 2. In some embodiments, the smallest area of the cross-section of the first opening of the sheathing 2 is usually different from the smallest area of the cross-section of the second opening. However, in some embodiments, the areas of the cross-sections of the first and the second opening of the sheathing 2 are identical. In some embodiments, the smallest areas of the cross-sections of the first and the second opening of the sheathing 2 are identical.

A surround body 5 is arranged in the direction of the second end 4b of the sheathing 2. The surround body 5 adjoins the rod-shaped insulating body 3 and the sheathing 2. The surround body 5 has a first surface which adjoins a surface of the inner side of the sheathing 2. The surround body 5 has a second surface which adjoins a surface of the outer side of the rod-shaped insulating body 3. It is preferred that the first surface of the surround body 5 is cylindrical. In some embodiments, the second surface of the surround body 5 is cylindrical. In some embodiments, the first and the second surface of the surround body 5 are cylindrical.

A smallest spacing between the first and the second surface of the surround body 5 influences a spacing of the rod-shaped insulating body 3 from the sheathing 2. The spacing between the rod-shaped insulating body 3 and the sheathing 2 can be for example less than ten millimeters or less than five millimeters. A spacing between the rod-shaped insulating body 3 and the sheathing 3 facilitates the production of the arrangement. A spacing between the rod-shaped insulating body 3 and the sheathing 2 renders possible a (limited) thermal insulation of the insulating body 3 and the supply lines 1a, 1b, 1c from the sheathing 2. Such a (limited) thermal insulation may be advantageous with respect to the increased temperatures in the exhaust gas duct of a combustion apparatus.

In some embodiments, the surround body 5 is at least in part made of a casting compound. For example, the surround body 5 can be produced wholly or in part from a heat-resistant epoxy resin. In a different embodiment, the surround body 5 is made of polytetrafluoroethylene. If a surround body 5 comprises a cast body this may contribute to a gas-tight arrangement.

In some embodiments, the surround body 5 or parts of the surround body 5 are produced using an additive production method such as the three-dimensional pressure. The surround body 5 or parts of the surround body 5 can be produced by selective laser sintering.

In some embodiments, the surround body 5 has a specific electrical resistance p of at least one megaohm·centimeter at temperatures of 393 Kelvin:

$$ρ > 1 MΩ \cdot cm at 393 Kelvin$$

The specific electrical resistance p at temperatures of 393 Kelvin may be greater than five megaohm·centimeters:

$$ρ > 5 MΩ \cdot cm at 393 Kelvin$$

Furthermore, the specific electrical resistance p at temperatures of 393 Kelvin may be greater than ten megaohm·centimeters:

$$ρ > 10 MΩ \cdot cm at 393 Kelvin$$

High specific resistances enable sufficient electrical insulation between the supply lines 1a, 1b, 1c. Any signal obtained from a sensor element that is connected to the supply lines 1a, 1b, 1c is therefore not distorted to any great extent.

The surround body 5 has at least one passage for one of the supply lines 1a, 1b, 1c. In some embodiments, the surround body 5 has at least as many passages as the arrangement has supply lines 1a, 1b, 1c. Moreover, the number of passages through the surround body 5 can exceed the number of supply lines 1a, 1b, 1c. With a number of passages through the insulating body 3 that exceeds the number of supply lines 1a, 1b, 1c, further supply lines can be added at a later point in time. Moreover, it is also possible for the same surround body 5 to be used for embodiments with different numbers of supply lines 1a, 1b, 1c. The use of a surround body 5 with a predetermined number of passages, limits the number of variants. Consequently, the probability that one of the variants fails during the course of their operation reduces.

The passage or passages through the surround body 5 each have a diameter. For example, the passage or passages through the surround body 5 can have a diameter of at least 0.6 millimeters or of at least 1.1 millimeters. One or multiple passages through the surround body 5 which have a diameter greater than 1.5 millimeters are also possible.

In the case of multiple passages through the surround body 5, at least two passages through the surround body 5 may have the same diameter. In some embodiments, all passages through the surround body 5 have the same diameter. Identical diameters of the passages through the surround body 5 lead to a small number of variants. Consequently, the probability that one of the variants fails during the course of their operation reduces. In addition, the outlay for the production of the surround body 5 reduces.

In some embodiments, the rod-shaped insulating body 3 and the surround body 5 have the same number of 1 passages. In some embodiments, the passages through the rod-shaped insulating body 3 and the surround body 5 also have the same or essentially the same diameter. Essentially the same diameter means here that the diameters are identical apart from manufacturing tolerances. In this case, the passages through the rod-shaped insulating body 3 and through the surround body 5 are arranged in such a manner that at least one supply line 1a runs directly through the arrangement. In some embodiments, the respective passages are arranged in such a manner that all supply lines 1a, 1b 1c run directly through the arrangement. A direct run through the arrangement renders possible a straight line passage through the rod-shaped insulating body 3 and through the surround body 5 without kinks.

Furthermore, the passages through the rod-shaped insulating body 3 and the surround body 5 can also have similar diameters. For example, the passages through the surround body 5 can each be a little wider than the passages through the rod-shaped insulating body 3 in order to facilitate a simpler assembly. In this case, the passages through the rod-shaped insulating body 3 and through the surround body 5 are arranged in such a manner that at least one supply line 1a runs directly through the arrangement. In some embodiments, the respective passages are arranged in such a manner that all supply lines 1a, 1b 1c run directly through the arrangement. A direct run through the arrangement renders possible a straight line passage through the rod-shaped insulating body 3 and through the surround body 5 without kinks.

At least one passage through the surround body 5 can comprise a hole, for example a hole with a round cross-section. In some embodiments, at least one passage through the surround body 5 can be a hole, for example a hole with a round cross-section. Moreover, all passages through the surround body 5 can each comprise a hole, for example a hole with a round cross-section. In addition, all passages through the surround body 5 can each be a hole, for example a hole with a round cross-section. The design of the passages through the surround body 5 as holes renders it possible to use standard tools for producing the surround body 5.

A recess for a first ring 6a, 6b is provided in the surround body 5. The first ring 6a, 6b surrounds the rod-shaped insulating body 3. The first ring 6a, 6b lies against the rod-shaped insulating body 3. Moreover, the first ring 6a, 6b lies against the sheathing 2. Consequently, the first ring 6a, 6b lies both against the rod-shaped insulating body 3 and also the sheathing 2. The first ring 6a, 6b is located between the rod-shaped insulating body 3 and the sheathing 2. For example, the first ring 6a, 6b can comprise a section. The section of the first ring 6a, 6b can run in a first groove or in a first furrow which is formed by the surround body 5 and/or the sheathing 2.

In some embodiments, the first ring 6a, 6b is an O-ring, such as for example a temperature-resistant O-ring. In particular, the O-ring can be made of a silicone polymer. In some embodiments, the first ring 6a, 6b is a retaining ring, such as for example a temperature-resistant retaining ring. In particular, the retaining ring can be made of a silicone polymer. In some embodiments, the first ring 6a, 6b is an O-ring and retaining ring such as for example a temperature-resistant O-ring and retaining ring. In particular, the O-ring and retaining ring can be made of a silicone polymer.

The first ring 6a, 6b can also be a seal ring. This means that the first ring 6a, 6b helps to ensure that no gases are conducted between the ends 4a, 4b. For example, the first ring 6a, 6b helps to ensure that no combustion gases are conducted between the ends 4a, 4b of the arrangement. In some embodiments, the first ring 6a, 6b is both O-ring and also seal ring. In a further special embodiment, the first ring 6a, 6b is both O-ring and also retaining ring and also seal ring.

The first ring 6a, 6b is arranged in the proximity of the second end 4b of the arrangement. This means that the first ring 6a, 6b has a smallest spacing from the first end 4a and a smallest spacing from the second end 4b. In this case, the smallest spacing of the first ring 6a, 6b from the first end 4a is greater than its smallest spacing from the second end 4b.

A second ring 7a, 7b is arranged closer to the first end 4a. This means that the second ring 7a, 7b has a smallest spacing from the first end 4a and a smallest spacing from the second end 4b. In this case, the smallest spacing of the second ring 7a, 7b from the first end 4a is smaller than its smallest spacing from the second end 4b. The smallest spacing of the second ring 7a, 7b from the first end 4a is smaller than the spacing of the first ring 6a, 6b from the same end 4a. The smallest spacing of the second ring 7a, 7b from the second end 4a is greater than the spacing of the first ring 6a, 6b from the same end 4b.

The second ring 7a, 7b surrounds the rod-shaped insulating body 3. The second ring 7a, 7b lies against the rod-shaped insulating body 3. Moreover, the second ring 7a, 7b lies against the sheathing 2. Consequently, the second ring 7a, 7b lies both against the rod-shaped insulating body 3 and also the sheathing 2. For example, the second ring 7a, 7b can comprise a section. The section of the second ring 7a, 7b can run in a second groove or in a second furrow which is formed by the sheathing 2. The second ring 7a, 7b is accordingly located between the rod-shaped insulating body 3 and the sheathing 2.

In some embodiments, the second ring 7a, 7b is a snap ring, such as for example a temperature-resistant snap ring. In particular, the snap ring can be made of a corrosion-resistant steel. In some embodiments, the second ring 7a, 7b is a retaining ring, such as for example a temperature-resistant retaining ring. In particular, the retaining ring can be made of a corrosion-resistant steel. In some embodiments, the second ring 7a, 7b is a snap ring and retaining ring such as for example a temperature-resistant snap ring and retaining ring. In particular, the snap ring and retaining ring can be made of a corrosion-resistant steel.

The first ring 6a, 6b and the second ring 7a, 7b help to secure the rod-shaped insulating body 3 against the sheathing 2. In some embodiments, the first ring 6a, 6b and the second ring 7a, 7b render possible the aforementioned smallest spacing between the rod-shaped insulating body 3 and the sheathing 2. Moreover, the first ring 6a, 6b and the second ring 7a, 7b prevent a lateral movement of the rod-shaped insulating body 3 between the ends 4a, 4b.

The rod-shaped insulating body 3 and the sheathing 2 can have different coefficients of thermal expansion. By way of example, the rod-shaped insulating body 3 can have a coefficient of thermal expansion α between 2·10⁻⁶/Kelvin and 10·10⁻⁶/Kelvin in the range between 313 Kelvin and 673 Kelvin:

$$2 \cdot {10}^{- 6} / Kelvin < α < 10 \cdot {10}^{- 6} / Kelvin$$

In particular, the rod-shaped insulating body 3 can have a coefficient of thermal expansion α between 3·10⁻⁶/Kelvin and 9·10⁻⁶/Kelvin in the range between 313 Kelvin and 673 Kelvin:

$$3 \cdot {10}^{- 6} / Kelvin < α < 9 \cdot {10}^{- 6} / Kelvin$$

In some embodiments, the rod-shaped insulating body 3 can have a coefficient of thermal expansion α between 4·10⁻⁶/Kelvin and 8·10⁻⁶/Kelvin in the range between 313 Kelvin and 673 Kelvin:

$$4 \cdot {10}^{- 6} / Kelvin < α < 8 \cdot {10}^{- 6} / Kelvin$$

The aforementioned coefficients of thermal expansion α relate to an axis which is defined by at least one of the supply lines 1a, 1b, 1c. This means that the coefficients of thermal expansion are axial coefficients along the aforementioned axis.

In some embodiments, the sheathing 2 can have a coefficient of thermal expansion α between 10·10⁻⁶/Kelvin and 20·10⁻⁶/Kelvin at 293 Kelvin:

$$10 \cdot {10}^{- 6} / Kelvin < α < 20 \cdot {10}^{- 6} / Kelvin$$

In particular, the sheathing 2 can have a coefficient of thermal expansion α between 11·10⁻⁶/Kelvin and 19·10⁻⁶/Kelvin at 293 Kelvin:

$$11 \cdot {10}^{- 6} / Kelvin < α < 19 \cdot {10}^{- 6} / Kelvin$$

In some embodiments, the sheathing 2 can have a coefficient of thermal expansion α between 11·10⁻⁶/Kelvin and 18·10⁻⁶/Kelvin at 293 Kelvin:

$$11 \cdot {10}^{- 6} / Kelvin < α < 18 \cdot {10}^{- 6} / Kelvin$$

The aforementioned coefficients of thermal expansion α relate to an axis which is defined by at least one of the supply lines 1a, 1b, 1c. This means that the coefficients of thermal expansion xx are axial coefficients along the aforementioned axis.

As a result of the different coefficients of thermal expansion of the sheathing 2 and the rod-shaped insulating body 3, mechanical stresses can occur in the arrangement. The problem of mechanical stresses is exacerbated by the wide range of temperatures to which the arrangement is exposed during operation. The first 6a, 6b and the second ring 7a, 7b support the rod-shaped insulating body 3 relative to the sheathing 2 in such a manner that cracks due to these mechanical stresses are avoided.

In some embodiments, a third ring 8a, 8b is arranged even closer to the first end 4a. This means that the third ring 8a, 8b has a smallest spacing from the first end 4a and a smallest spacing from the second end 4b. In this case, the smallest spacing of the third ring 8a, 8b from the first end 4a is smaller than its smallest spacing from the second end 4b. The smallest spacing of the third ring 8a, 8b from the first end 4a is smaller than the spacing of the first ring 6a, 6b from the same end 4a. The smallest spacing of the third ring 8a, 8b from the second end 4a is greater than the spacing of the first ring 6a, 6b from the same end 4b.

1. An arrangement comprising:
a first electrically conductive supply line;
a sheathing; and
a rod-shaped insulating body comprising an electrically insulating and temperature-resistant material;
wherein the sheathing has a first end and a second end, the first end opposite the second end;
the sheathing has a first opening at the first end and a second opening at the second end;
the first supply line runs from the first end to the second end through the arrangement and defines an axis;
the rod-shaped insulating body comprises passage therethrough and a first section of the supply line runs in the passage;
the first supply line has a first length defined between the first opening and the second opening;
the first section of the supply line has a second length defined in the passage;
the rod-shaped insulating body comprises an outer surface radially outwards from the axis and the sheathing comprises an inner surface outwards from the axis, wherein the outer surface of the rod-shaped insulating body and the inner surface of the sheathing both run parallel to the axis, so that the outer surface of the rod-shaped insulating body and the inner surface of the sheathing lie opposite one another;
a gap with a size and arranged between the outer surface of the rod-shaped insulating body and the inner surface of the sheathing; and
spacers providing a spacing between the rod-shaped insulating body and the sheathing, wherein the spacers define the size of the gap;
wherein the second length is smaller than the first length; and
wherein the spacers comprise a first ring running along a first closed curve around the rod-shaped insulating body and adjoins the rod-shaped insulating body and adjoins the sheathing.

2. The arrangement as claimed in claim 1, wherein the spacers provide a spacing between the outer surface of the rod-shaped insulating body and the inner surface of the sheathing and define the size of the gap.

3. The arrangement as claimed in claim 1, wherein the first supply line runs from the first end to the second end in a straight line through the arrangement and defines the axis.

4. The arrangement as claimed in claim 1, wherein the spacers comprise a surround body with a first section running perpendicular to the axis and adjoining the rod-shaped insulating body, and at least one second section running parallel to the axis and adjoining the sheathing.

5. The arrangement as claimed in claim 4, wherein the surround body comprises a passage and a second section of the supply line in the passage runs through the surround body.

6. The arrangement as claimed in claim 4, wherein the surround body can be plugged onto the rod-shaped insulating body in the axial direction outwards from the axis.

7. The arrangement as claimed in claim 4, wherein the surround body can be plugged into the sheathing in the axial direction outwards from the axis.

8. The arrangement as claimed in claim 4, wherein:
the surround body has a first spacing to the first end and a second spacing to the second end; and
the first spacing of the surround body from the first end is greater than the second spacing of the surround body from the second end.

9. The arrangement as claimed in claim 4, wherein the rod-shaped insulating body and the surround body are each electrical insulators and at temperatures of 393 Kelvin each have a specific electrical resistance p of at least one megaohm·centimeter.

10. The arrangement as claimed in claim 1, wherein:
the first ring has a first spacing to the first end and a second spacing to the second end; and
the first spacing of the first ring from the first end is greater than the second spacing of the first ring from the second end.

11. The arrangement as claimed in claim 4, wherein the surround body comprises a recess and the first ring comprises a section which is arranged and/or runs in the recess of the surround body.

12. The arrangement as claimed in claim 1, wherein:
the spacers comprise a second ring running along a second closed curve around the rod-shaped insulating body and adjoining the rod-shaped insulating body and adjoining the sheathing and is different from the first ring;
the second ring has a first spacing to the first end and a second spacing to the second end; and
the first spacing of the second ring from the first end is smaller than the second spacing of the second ring from the second end.

13. A combustion apparatus comprising:
a combustion chamber; and
a structure selected from: an exhaust gas stack, a flue gas stack, and a chimney;
wherein the structure is in fluid communication with the combustion chamber;
a first electrically conductive supply line;
a sheathing; and
a rod-shaped insulating body comprising an electrically insulating and temperature-resistant material;
wherein the sheathing has a first end and a second end, the first end opposite the second end;
the sheathing has a first opening at the first end and a second opening at the second end;
the first supply line runs from the first end to the second end through the arrangement and defines an axis;
the rod-shaped insulating body comprises a passage therethrough and a first section of the supply line runs in the passage;
the first supply line has a first length defined between the first opening and the second opening;
the first section of the supply line has a second length defined in the passage;
the rod-shaped insulating body comprises an outer surface radially outwards from the axis and the sheathing comprises an inner surface outwards from the axis, wherein the outer surface of the rod-shaped insulating body and the inner surface of the sheathing both run parallel to the axis, so that the outer surface of the rod-shaped insulating body and the inner surface of the sheathing lie opposite one another;
a gap with a size and arranged between the outer surface of the rod-shaped insulating body and the inner surface of the sheathing; and
spacers providing a spacing between the rod-shaped insulating body and the sheathing, wherein the spacers define the size of the gap;
wherein the second length is smaller than the first length; and
wherein the spacers comprise a first ring running along a first closed curve around the rod-shaped insulating body and adjoins the rod-shaped insulating body and adjoins the sheathing; and
wherein at least one section of the arrangement is arranged within the structure.

14. The combustion apparatus as claimed in claim 13, wherein:
the structure comprises an outer wall; and
the sheathing of the arrangement is guided through the outer wall in such a manner that the at least one section of the arrangement protrudes into the structure.

15. An arrangement comprising:
a first electrically conductive supply line,
a sheathing; and
a rod-shaped insulating body comprising an electrically insulating and temperature-resistant material;
wherein the sheathing has a first end and a second end and the first end is different from the second end and the first end is opposite the second end and the sheathing has a first opening at the first end and a second opening at the second end;
wherein the first supply line runs from the first end to the second end through the arrangement and defines an axis;
wherein the rod-shaped insulating body comprises a passage through the rod-shaped insulating body and a first section of the supply line runs in the passage through the rod-shaped insulating body;
wherein the first supply line has a first length between the first opening and the second opening;
wherein the first section of the supply line has a second length in the passage through the rod-shaped insulating body and the second length is smaller than the first length;
wherein the rod-shaped insulating body comprises an outer surface radially outwards from the axis and the sheathing comprises an inner surface radially outwards from the axis, wherein the outer surface of the rod-shaped insulating body and the inner surface of the sheathing each run parallel to the axis, so that the outer surface of the rod-shaped insulating body and the inner surface of the sheathing lie opposite one another;
wherein the arrangement comprises a gap which has a size and is arranged between the outer surface of the rod-shaped insulating body and the inner surface of the sheathing, and comprises spacers so as to provide a spacing between the rod-shaped insulating body and the sheathing, wherein the spacers define the size of the gap;
wherein the spacers comprise a surround body, and
wherein the surround body comprises at least one first section, which runs perpendicular to the axis and adjoins the rod-shaped insulating body, and at least one second section, which runs parallel to the axis and adjoins the sheathing.
